(19) United States
(12) Patent Application Publication
(10) Pub. No.: US 2014/0366885 A1
(43) Pub. Date: Dec. 18, 2014

(54) PATIENT INTERFACE DEVICE WITH AUTOMATICALLY TILTING CUSHION

(71) Applicants: Richard Thomas Haibach, Verona, PA (US); Richard Andrew Sofranko, Finleyville, PA (US)

(72) Inventors: Richard Thomas Haibach, Verona, PA (US); Richard Andrew Sofranko, Finleyville, PA (US)

(73) Assignee: KONINKLIJKE PHILIPS N.V., EINDHOVEN (NL)

(21) Appl. No.: 14/345,453

(22) PCT Filed: Sep. 10, 2012
(86) PCT No.: PCT/IB2012/054688

§ 371 Date: Mar. 18, 2014

Related U.S. Application Data
(60) Provisional application No. 61/537,737, filed on Sep. 22, 2011.

(51) Int. Cl. A61M 16/06

(52) U.S. Cl. CPC A61M 16/0622; A61M 16/0666; A61M 16/0683; A61M 2016/0661; A61M 2205/0216
USPC 128/206.24

(57) ABSTRACT

A method of adjusting the fit of a patient interface device having a nasal cushion rotatably coupled to frame member includes providing resistance to rotation of the nasal cushion assembly in a first direction toward a face of a patient using a rotational resistance mechanism provided between the nasal cushion and the frame member, and responsive to a force being applied to the nasal cushion by a nose of the patient, allowing the nasal cushion to rotate relative to the frame member in the first direction against the resistance to a tilted position, wherein in the tilted position the rotational resistance mechanism biases the cushion assembly in favor of rotation in a second direction opposite the first direction.

### CROSS-REFERENCE TO RELATED APPLICATIONS

This patent application claims the priority benefit under 35 U.S.C. §119(e) of U.S. Provisional Application No. 61/537,737 filed on Sep. 22, 2011, the contents of which are herein incorporated by reference.

### BACKGROUND OF THE INVENTION

1. Field of the Invention

The present invention relates to patient interface devices for communicating a flow of gas with an airway of a user, and, in particular, to a patient interface device including a nasal cushion structured to tilt to automatically to adjust to different patient facial geometries.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

For such patient interface devices, a key engineering challenge is to balance patient comfort against mask stability and mask to face seal. This is particularly true in the case of treatment of OSA, where such patient interface devices are typically worn for an extended period of time. As a patient changes sleeping positions through the course of the night, masks tend to become dislodged, and the seal can be broken. A dislodged mask can be stabilized by increasing strapping force, but increased strapping force tends to reduce patient comfort. This design conflict is further complicated by the widely varying facial geometries that a given mask design needs to accommodate. One area where facial geometries vary a great deal is the angle of the base of the nose (known as the nasolabial angle). Because this angle varies so greatly, the optimum cushion tilt varies from patient to patient. As a result, the ability to accommodate a wide range of patient nose geometries is important in terms of seal and comfort of a nasal cushion type patient interface device.

One way to account for such different nose geometries is to include a manual tilt adjustment mechanism in the patient interface device wherein the angle of tilt of the mask component can be adjusted manually to different positions. A manual adjustment method, however, has the potential to cause confusion for the patient, which can lead to frustration and possibly reduce the effectiveness of the patient interface device.

### SUMMARY OF THE INVENTION

Accordingly, it is an object of the present invention to provide a patient interface device that overcomes the shortcomings of conventional patient interface devices. This object is achieved according to one embodiment of the present invention by providing a system that includes a patient interface device having a mechanism to allow the cushion assembly of the device to be automatically tilted to accommodate different patient nose geometries.

In one embodiment, a patient interface device is provided that includes a cushion assembly including a nasal cushion, a first post member extending from a first side of the cushion assembly and a second post member extending from a second side of the cushion assembly, and a frame member having a first arm and a second arm, wherein the first post member is rotatably coupled to the first arm and the second post member is rotatably coupled to the second arm such that the cushion assembly is rotatable with respect to the frame member about an axis extending through the first post member and the second post member. The patient interface device also includes a rotational resistance mechanism coupled to the cushion assembly and the frame member, the rotational resistance mechanism providing resistance to rotation of the cushion assembly in a first direction toward a face of a patient when the patient interface device is donned by the patient and tending to bias rotation of the cushion assembly in a second direction opposite the first direction responsive to the cushion assembly being rotated in the first direction.

In another embodiment, a patient interface device is provided that includes a nasal cushion, a frame member having a central portion positioned below the nasal cushion, a first arm extending from a first side of the central portion and a second arm extending from a second side of the central portion, and one or more spring members coupled to and extending from a surface of the nasal cushion. A distal end of each of the one or more spring members is coupled to the central portion of the frame member, the one or more spring members providing resistance to rotation of the nasal cushion in a first direction toward a face of a patient when the patient interface device is donned by the patient and tending to bias rotation of the cushion assembly in a second direction opposite the first direction responsive to the cushion assembly being rotated in the first direction.

In still another embodiment, a method of adjusting a fit of a patient interface device having a nasal cushion rotatably coupled to frame member is provided that includes providing resistance to rotation of the nasal cushion assembly in a first direction toward a face of a patient using a rotational resistance mechanism provided between the nasal cushion and the frame member, and responsive to a force being applied to the nasal cushion by a nose of the patient, allowing the nasal cushion to rotate relative to the frame member in the first direction against the resistance to a tilted position, wherein in the tilted position the rotational resistance mechanism biases the cushion assembly in favor of rotation in a second direction opposite the first direction.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1 and 2 are schematic diagrams of a patient interface device adapted to provide a regimen of respiratory therapy to a patient according to one exemplary embodiment of the present invention;

FIG. 3 is a schematic diagram of a patient interface device adapted to provide a regimen of respiratory therapy to a patient according to another exemplary embodiment of the present invention;

FIGS. 4A and 4B are cross-sectional views of the patient interface device of FIG. 3 illustrating the operation thereof;

FIG. 5 is a schematic diagram of a patient interface device adapted to provide a regimen of respiratory therapy to a patient according to another alternative exemplary embodiment of the present invention;

FIGS. 6A, 6B and 6C are cross-sectional views of the patient interface device of FIG. 5 illustrating the operation thereof;

FIG. 7 is a schematic diagram of a patient interface device adapted to provide a regimen of respiratory therapy to a patient according to a further alternative exemplary embodiment of the present invention;

FIGS. 8A, 8B and 8C are cross-sectional views of the patient interface device of FIG. 7 illustrating the operation thereof;

FIG. 9 is a schematic diagram of a patient interface device adapted to provide a regimen of respiratory therapy to a patient according to still a further alternative exemplary embodiment of the present invention;

FIGS. 10A and 10B are cross-sectional views of the patient interface device of FIG. 9 illustrating the operation thereof;

FIG. 11 is a schematic diagram of a patient interface device adapted to provide a regimen of respiratory therapy to a patient according to yet another alternative exemplary embodiment of the present invention; and

FIGS. 12A and 12B are cross-sectional views of the patient interface device of FIG. 11 illustrating the operation thereof.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

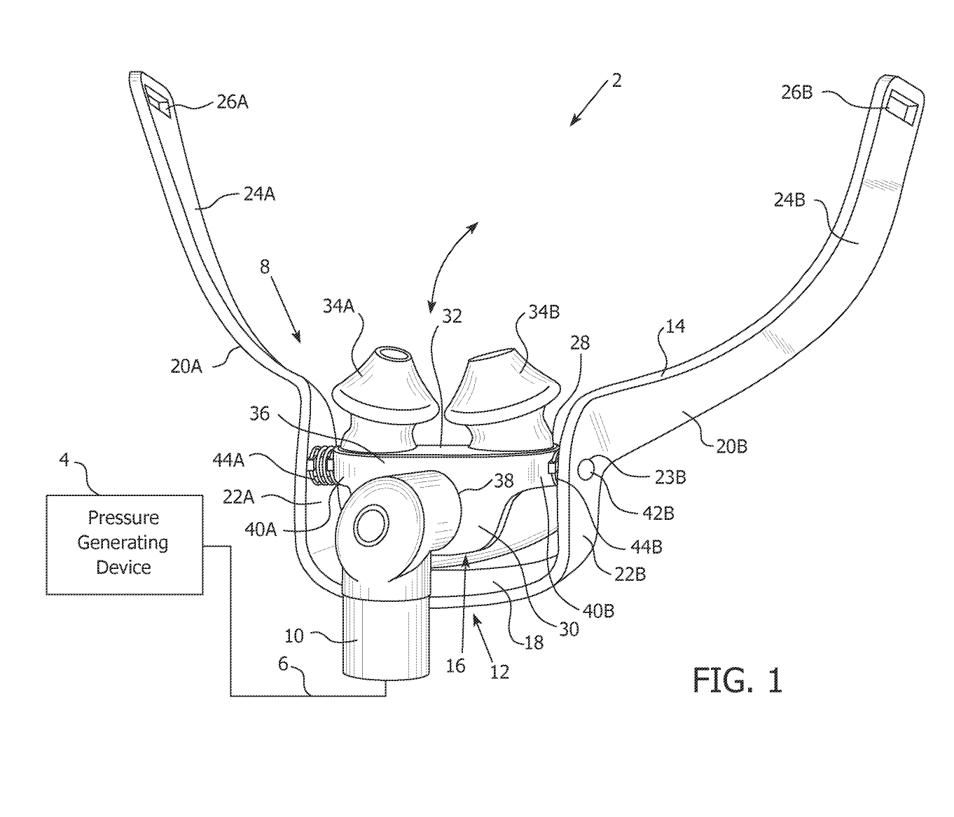

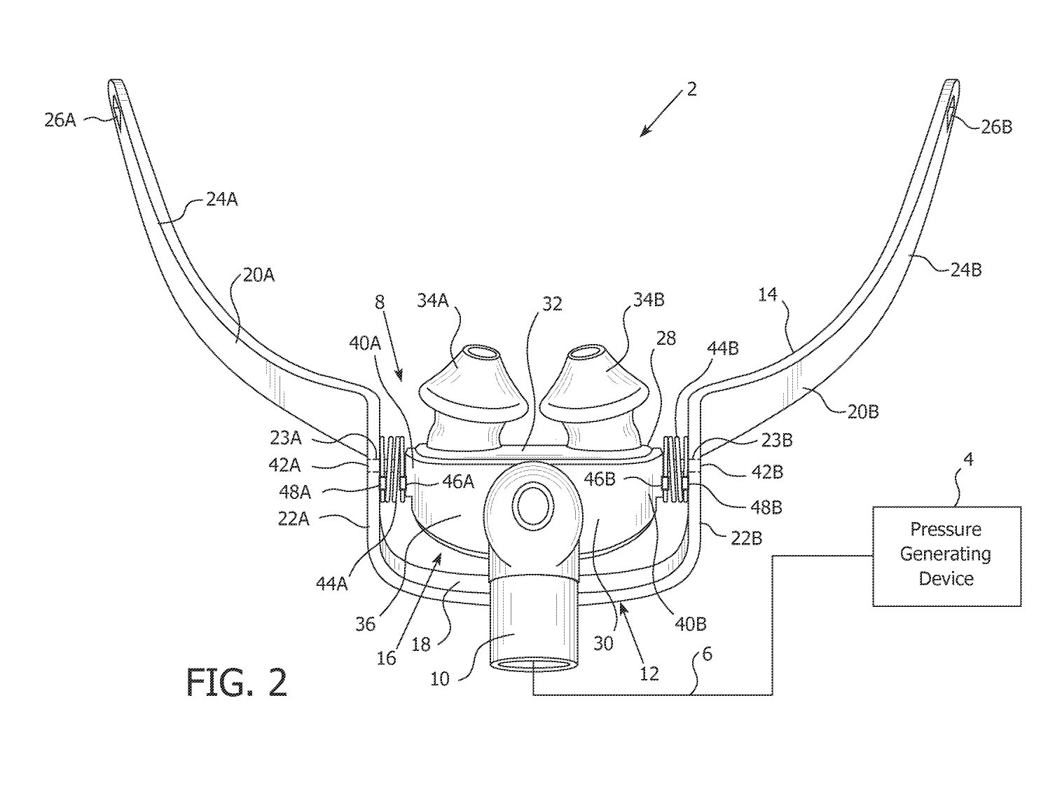

A system 2 adapted to provide a regimen of respiratory therapy to a patient according to one exemplary embodiment is generally shown in FIGS. 1 and 2. System 2 includes a pressure generating device 4, a delivery conduit 6, and a patient interface device 8 having a fluid coupling conduit 10 (patient interface device 8 is shown in isometric view in FIG. 1 and in front elevational view in FIG. 2). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

As seen in FIGS. 1 and 2, patient interface device 8 includes a patient sealing assembly 12 that facilitates the delivery of the flow of breathing gas to the airway of a patient. Patient sealing assembly 12 includes a frame member 14 having a cushion assembly 16 rotatably coupled thereto in the manner described in greater detail below.

In the illustrated embodiment, frame member 14 is made of a rigid or semi-rigid material, such as, without limitation, an injection molded thermoplastic or silicone. Frame member 14 includes a generally flat central portion 18 positioned beneath cushion assembly 16. Frame member 14 further includes arms 20A and 20B positioned on opposite sides of central portion 18. More specifically, arms 20A and 20B each include a respective mounting portions 22A, 22B extending in a direction that is substantially normal to the top surface of central portion 18 on either side thereof, and extension portions 24A, 24B which extend form the distal end of mounting portions 22A, 22B. Each mounting portion 22A, 22B includes a pivot opening 23A, 23B extending therethough. The purpose of the pivot openings 23A, 23B is described elsewhere herein. In addition, each extension portion 24A, 24B includes a looped connector 26A, 26B structured to enable a headgear strap (not shown) to be attached to frame ember 14 in a known manner.

Cushion assembly 16 includes a nasal cushion 28 structurally and fluidly coupled to a sub-frame member 30. In the illustrated embodiment, nasal cushion 28 is a “pillows” style nasal cushion made of flexible, cushiony, elastomeric material, such as, without limitation, silicone, an appropriately soft thermoplastic elastomer, a closed cell foam, or any combination of such materials. As seen in FIGS. 1 and 2, the exemplary pillows style nasal cushion 28 includes a main body portion 32 having nasal prongs 34A and 34B extending from a top side thereof. Alternatively, nasal cushion 28 may be a “cradle” style nasal cushion that rests beneath and covers the patient's nares, or some other suitable nasal cushion configuration structured to engage the nose of the patient.

In addition, sub-frame member 30 is made of a rigid or semi-rigid material, such as, without limitation, an injection molded thermoplastic or silicone. Sub-frame member 30 includes a central support portion 36 having a central opening 38. As seen in FIGS. 1 and 2, fluid coupling conduit 10 is coupled to a front side of central support portion 36 through opening 38. As also seen in FIGS. 1 and 2, main body portion 32 of nasal cushion 28 is sealingly coupled to a rear side of central support portion 36 such that fluid coupling conduit is in fluid communication with the interior of nasal cushion 28 through opening 38. This configuration allows the flow of breathing gas from pressure generating device 4 to be communicated to nasal cushion 28, and then to the airway of a patient (through the patient's nares which are engaged by nasal prongs 34A, 34B).

Sub-frame member 30 further includes pivot mounting portions 40A, 40B extending from opposite sides thereof. Each pivot mounting portion 40A, 40B includes a post member 42A, 42B.

As seen in FIGS. 1 and 2, cushion assembly 16 is rotatably mounted to frame member 14 by inserting post members 42A, 42B through respective pivot openings 23A, 23B. When post members 42A, 42B are received in pivot openings 23A, 23B in this manner, cushion assembly 16 is able to rotate relative to frame member 14 about an axis through mounting portions 22A, 22B in a plane that is generally parallel to the extension portions 24A, 24B (i.e., toward and away from the patient's face as shown by the arrows in FIG. 1).

In addition, as seen in FIGS. 1 and 2, coiled spring members 44A and 44B are provided in between sub-frame member 30 and arms 20A and 20B on either side of sub-frame member 30. In particular, each spring member 44A, 44B includes a first terminal end 46A, 46B that is coupled to a respective pivot mounting portion 40A, 40B, and a second terminal end 48A, 48B that is coupled to a respective arm 20A, 20B. In this manner, coiled spring members 44A and 44B provide rotational resistance to cushion assembly 16 relative to frame member 14. More particularly, cushion assembly 16 will have a preset original base position wherein coiled spring members 44A and 44B are in a relaxed state (with no force being applied to cushion assembly 16). When a force is applied to nasal prongs 34A, 34B by the nose of the patient as a result of patient interface device 8 being donned by the patient, that force will work against the rotational resistance provided by spring members 44A, 44B and cause cushion assembly 16 to automatically rotate (tilt) relative to frame member 14 (about the axis described above) to a tilted/loaded position that will accommodate the particular nose geometry of the patient. When that force is removed, the bias of the spring members 44A, 44B will cause the cushion assembly 16 to automatically return to the original base position (unloaded). As a result, patient interface device 8 is able to automatically adjust to accommodate different patient facial geometries.

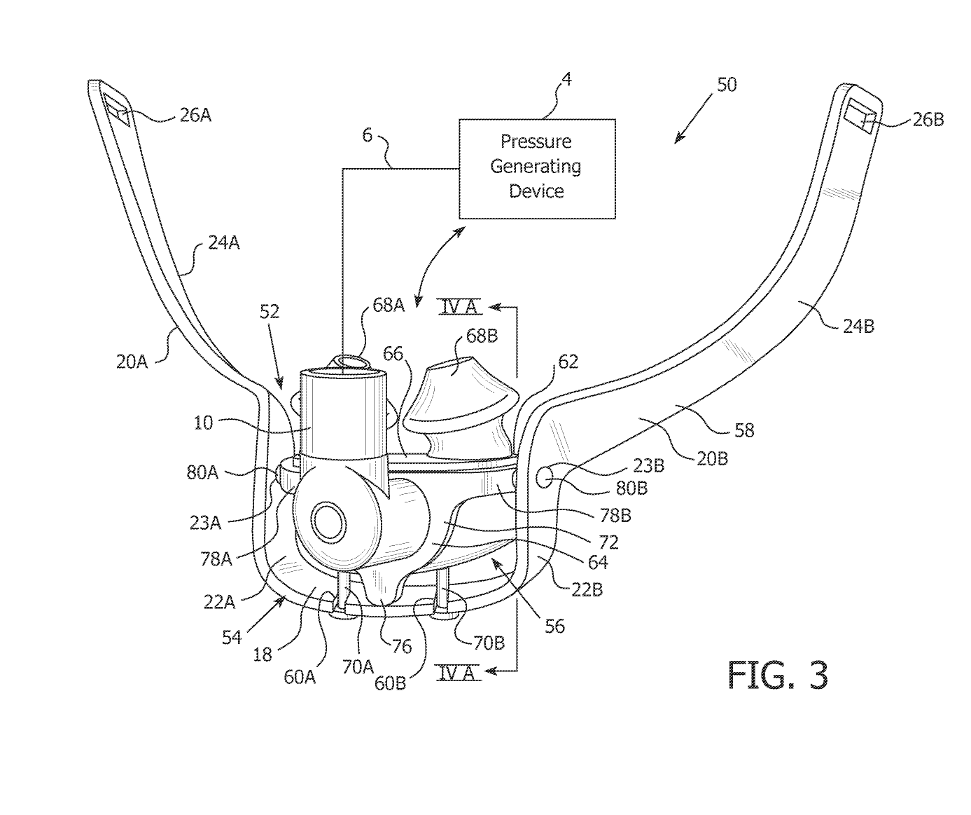

A system 50 adapted to provide a regimen of respiratory therapy to a patient according to an alternative exemplary embodiment is generally shown in FIG. 3. System 50 includes a number of the same components as system 2, and like components are labeled with like reference numerals.

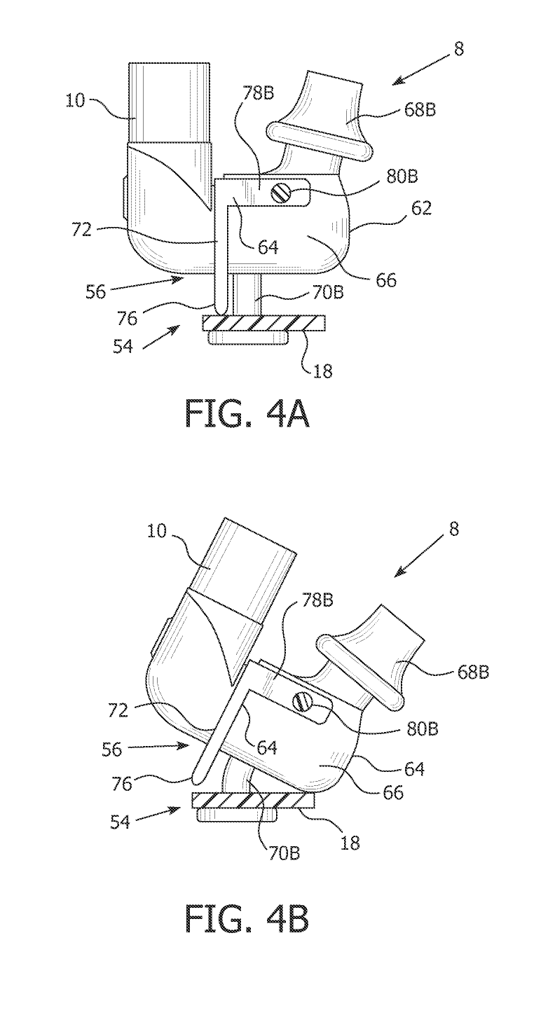

System 50 includes an alternative patient interface device 52 that is similar to patient interface device 8 (i.e., it includes fluid coupling conduit 10 and an automatically tilting cushion assembly/frame member combination). Patient interface device 52, however, includes an alternative patient sealing assembly 54 having an alternative cushion assembly 56 rotatably coupled to an alternative frame member 58 in the manner described in greater detail below. FIGS. 4A and 4B are cross-sectional views of a portion of alternative patient interface device 52 showing the operation of patient interface device 52 (described below).

As seen in FIG. 3, frame member 58 includes a number of the same parts as frame member 14. The central portion 18 of frame member 58, however, includes slots 60A, 60B formed therein, the function of which is described below.

Cushion assembly 56 includes a nasal cushion 62 structurally and fluidly coupled to a sub-frame member 64. In the illustrated embodiment, nasal cushion 62 is a “pillows” style nasal cushion made of flexible, cushiony, elastomeric material, such as, without limitation, silicone, an appropriately soft thermoplastic elastomer, a closed cell foam, or any combination of such materials. Alternatively, nasal cushion 62 may be a “cradle” style nasal cushion, or some other suitable nasal cushion configuration structured to engage the nose of the patient. As seen in FIGS. 3, 4A and 4B, the exemplary pillows style nasal cushion 62 includes a main body portion 66 having nasal prongs 68A and 68B extending from a top side thereof. In addition, nasal cushion 64 includes T-shaped elastomeric tensioner members 70A and 70B extending from a bottom side of main body portion 66. In the exemplary embodiment, elastomeric tensioner members 70A and 70B are integrally molded as part of nasal cushion 62, and may be made of the same material as main body portion 66 or a different material than main body portion 66.

In addition, sub-frame member 64, like sub-frame member 30 of FIGS. 1 and 2, is made of a rigid or semi-rigid material, such as, without limitation, an injection molded thermoplastic or silicone. Sub-frame member 64 includes a central support portion 72 having a central opening 74. As seen in FIGS. 3-4B, fluid coupling conduit 10 is coupled to a front side of central support portion 72 through opening 74. As also seen in FIGS. 3-4B, main body portion 66 of nasal cushion 62 is sealingly coupled to a rear side of central support portion 72 such that fluid coupling conduit 10 is in fluid communication with the interior of nasal cushion 62 through opening 74. This configuration allows the flow of breathing gas from pressure generating device 4 to be communicated to nasal cushion 62, and then to the airway of a patient (through the patient's nares which are engaged by nasal prongs 68A, 68B).

In addition, central support portion 72 of sub-frame member 64 further includes a stop member 76 extending downwardly therefrom. The purpose of stop member 76 is described below. Sub-frame member 64 also includes pivot mounting portions 78A, 78B extending from opposite sides thereof. Each pivot mounting portion 78A, 78B includes a post member 80A, 80B.

As seen in FIG. 3, cushion assembly 56 is rotatably mounted to frame member 58 by inserting post members 80A, 80B through respective pivot openings 23A, 23B. When post members 80A, 80B are received in pivot openings 23A, 23B in this manner, cushion assembly 56 is able to rotate relative to frame member 58 about an axis through mounting portions 22A, 22B in a plane that is generally parallel to the extension portions 24A, 24B (i.e., toward and away from the patient's face as shown by the arrows in FIG. 3).

In addition, elastomeric tensioner members 70A and 70B are each received within a respective slot 60A, 60B of central portion 18 of frame member 58. In this manner, elastomeric tensioner members 70A and 70B provide rotational resistance to cushion assembly 56 relative to frame member 58. More particularly, cushion assembly 56 will have a preset original base position wherein elastomeric tensioner members 70A and 70B are in an un-stretched, relaxed state (with no force being applied to cushion assembly) as seen in FIG. 4A. In this state, stop member 76 will engage a top surface of central portion 18. When a force is applied to nasal prongs 68A, 68B by the nose of the patient as a result of patient interface device 52 being donned by the patient, that force will work against the rotational resistance provided by elastomeric tensioner members 70A and 70B, causing them to stretch as shown in FIG. 4B, and will cause cushion assembly 56 to automatically rotate (tilt) relative to frame member 58 (about the axis described above) to a tilted/loaded position that will accommodate the particular nose geometry of the patient. When that force is removed, the bias of the elastomeric tensioner members 70A and 70B (wherein they will want to un-stretch), will cause the cushion assembly 56 to automatically return to the original base position (unloaded). Again, in this state, stop member 76 will engage the top surface of central portion 18 to prevent over rotation.

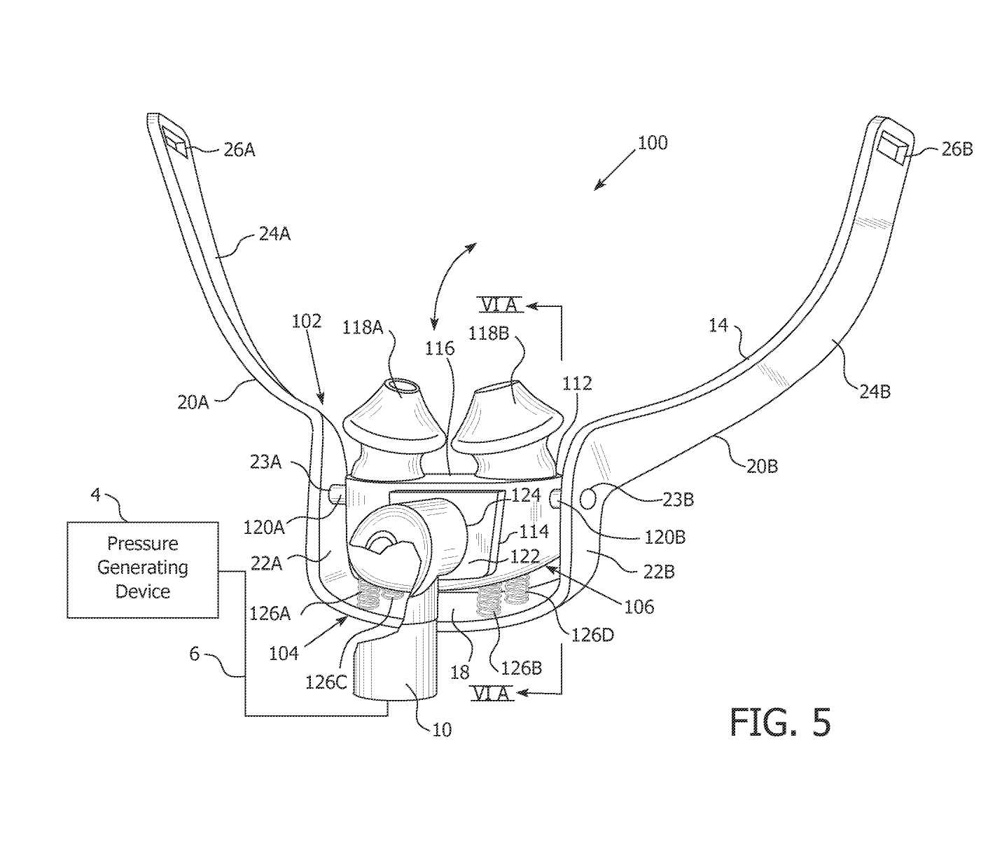

A system 100 adapted to provide a regimen of respiratory therapy to a patient according to another alternative exemplary embodiment is generally shown in FIG. 5. System 100 includes a number of the same components as system 2, and like components are labeled with like reference numerals.

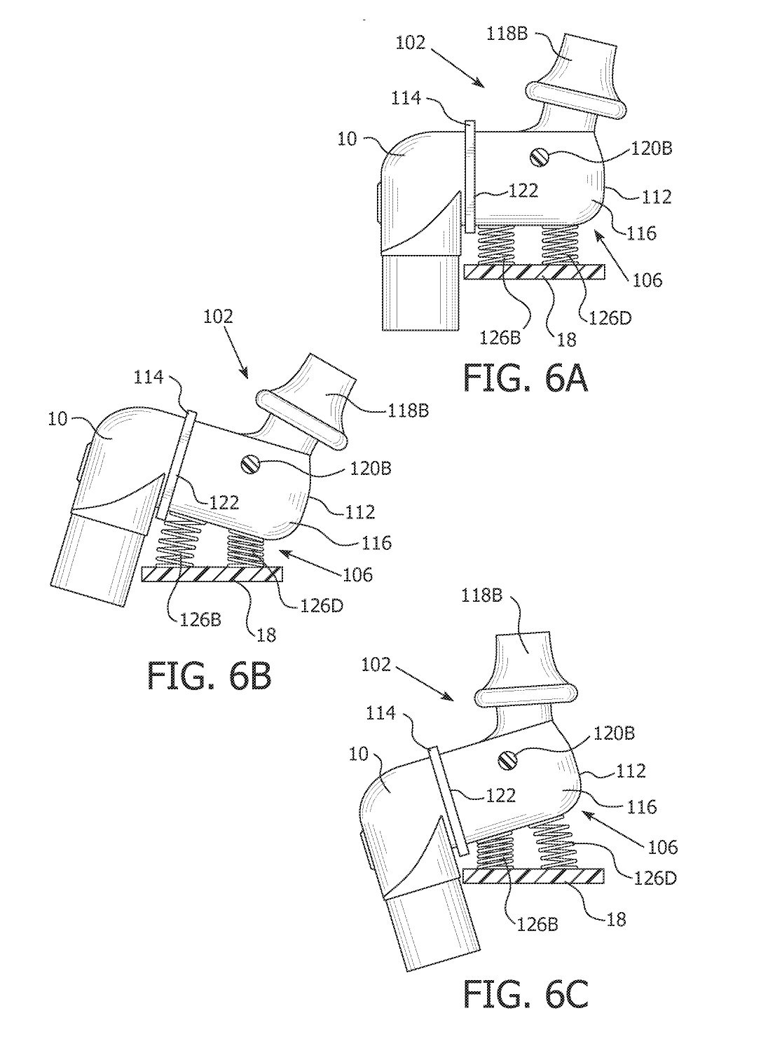

System 100 includes an alternative patient interface device 102 that is similar to patient interface device 8 (i.e., it includes fluid coupling conduit 10 (shown in partial cut-away form) and an automatically tilting cushion assembly/frame member combination). Patient interface device 102, however, includes an alternative patient sealing assembly 104 having an alternative cushion assembly 106 rotatably coupled to frame member 14 as described in connection with FIG. 1 in the manner described in greater detail below. FIGS. 6A, 6B and 6C are cross-sectional views of a portion of alternative patient interface device 102 showing the operation of patient interface device 102 (described below).

Cushion assembly 106 includes a nasal cushion 112 structurally and fluidly coupled to a sub-frame member 114. In the illustrated embodiment, nasal cushion 112 is a “pillows” style nasal cushion made of flexible, cushiony, elastomeric material, such as, without limitation, silicone, an appropriately soft thermoplastic elastomer, a closed cell foam, or any combination of such materials. Alternatively, nasal cushion 112 may be a “cradle” style nasal cushion, or some other suitable nasal cushion configuration structured to engage the nose of the patient. As seen in FIGS. 5-6C, the exemplary pillows style nasal cushion 112 includes a main body portion 116 having nasal prongs 118A and 118B extending from a top side thereof. In addition, nasal cushion 112 includes post members 120A and 120B extending from opposite sides of main body portion 116. In the exemplary embodiment, post members 120A and 120B are integrally molded as part of nasal cushion 112, and may be made of the same material as main body portion 116 or a different material than main body portion 116.

In addition, sub-frame member 114, like sub-frame member 30 of FIGS. 1 and 2, is made of a rigid or semi-rigid material, such as, without limitation, an injection molded thermoplastic or silicone. Sub-frame member 114 includes a central support portion 122 having a central opening 124. As seen in FIGS. 5-6C, fluid coupling conduit 10 is coupled to a front side of central support portion 122 through opening 124. As also seen in FIGS. 5-6C, main body portion 116 of nasal cushion 112 is sealingly coupled to a rear side of central support portion 122 such that fluid coupling conduit 10 is in fluid communication with the interior of nasal cushion 112 through opening 124. This configuration allows the flow of breathing gas from pressure generating device 4 to be communicated to nasal cushion 112, and then to the airway of a patient (through the patient's nares which are engaged by nasal prongs 118A, 118B).

Moreover, in this embodiment, a plurality of coiled (metal, e.g., steel) spring members 126 are provided in between the bottom surface of nasal cushion 112 and the top surface of central portion 18 of frame member 14. In the illustrated exemplary embodiment, front spring members 126A and 126B and rear spring member 126C and 126D are provided. Also, as seen in FIG. 5, cushion assembly 106 is rotatably mounted to frame member 14 by inserting post members 120A, 120B through respective pivot openings 23A, 23B. When post members 120A, 120B are received in pivot openings 23A, 23B in this manner, cushion assembly 106 is able to rotate relative to frame member 14 about an axis through mounting portions 22A, 22B in a plane that is generally parallel to the extension portions 24A, 24B (i.e., toward and away from the patient's face as shown by the arrows in FIG. 5).

In addition, spring members 126 provide rotational resistance to cushion assembly 116 relative to frame member 14. More particularly, cushion assembly 116 will have a preset original base position shown in FIGS. 5 and 6A (with no force being applied to cushion assembly 116). When a force is applied to nasal prongs 118A, 118B by the nose of the patient as a result of patient interface device 102 being donned by the patient, that force will work against the rotational resistance provided spring members 126, causing front spring members 126A, 126B to stretch and rear spring member 126C, 126D to compress and bend as shown in FIG. 6B, and will cause cushion assembly 106 to automatically rotate (tilt) relative to frame member 14 (about the axis described above) to a tilted/loaded position that will accommodate the particular nose geometry of the patient. When that force is removed, the bias of spring members 126 will cause the cushion assembly 106 to automatically tilt in the reverse direction (as shown in FIG. 6C) and ultimately return to the original base position of FIG. 6A.

In an alternative embodiment, sub-frame member 114 may further include a stop member extending downwardly therefrom to restrict the reverse rotation of cushion assembly 116. In another alternative embodiment, post members 120A, 120B may be omitted and instead sub-frame member 114 may include pivot mounting portions having post members (like pivot mounting portions 78A, 78B having post member 80A, 80B) extending from opposite sides thereof.

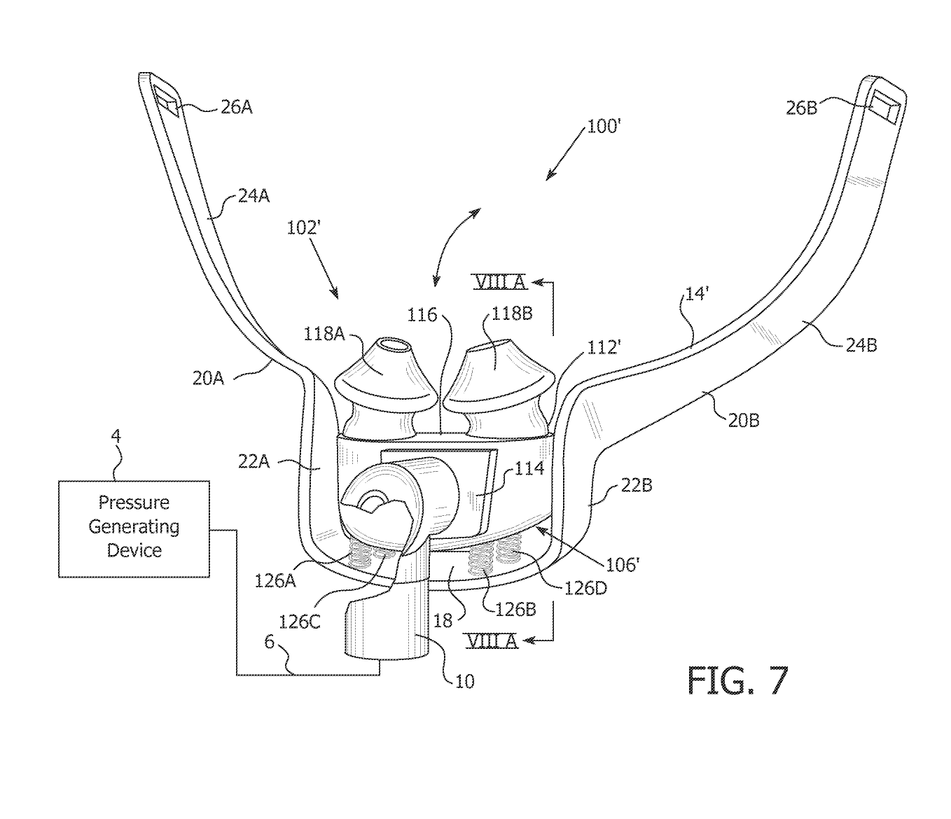

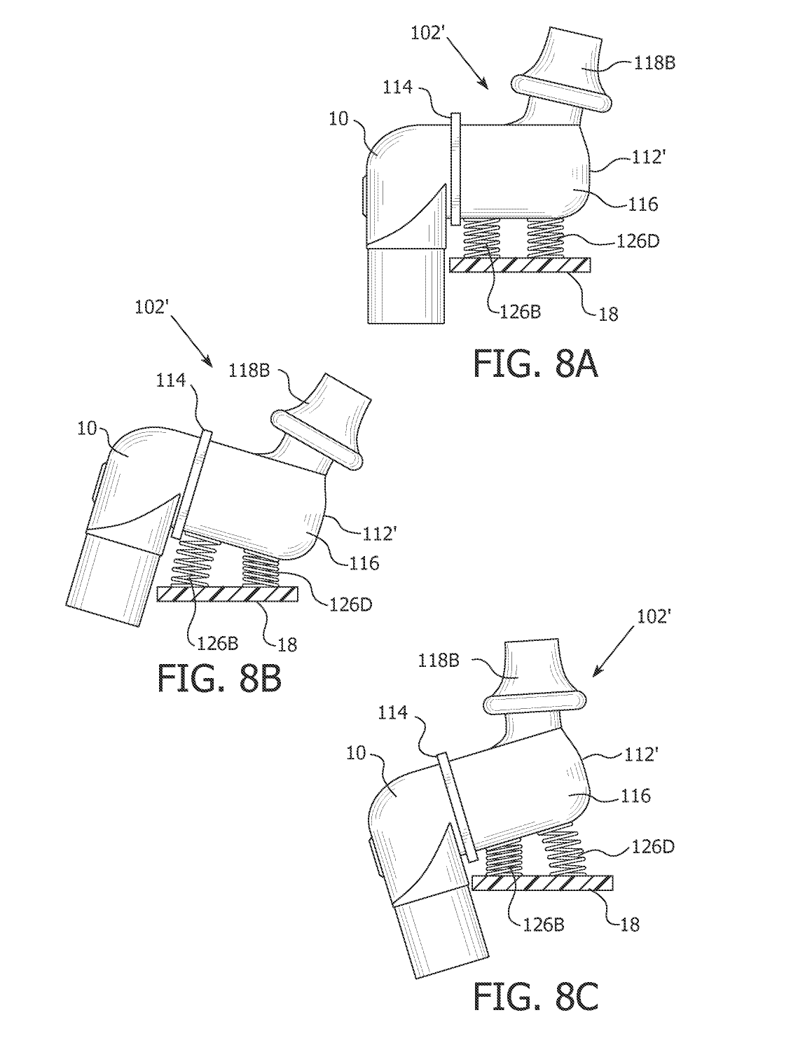

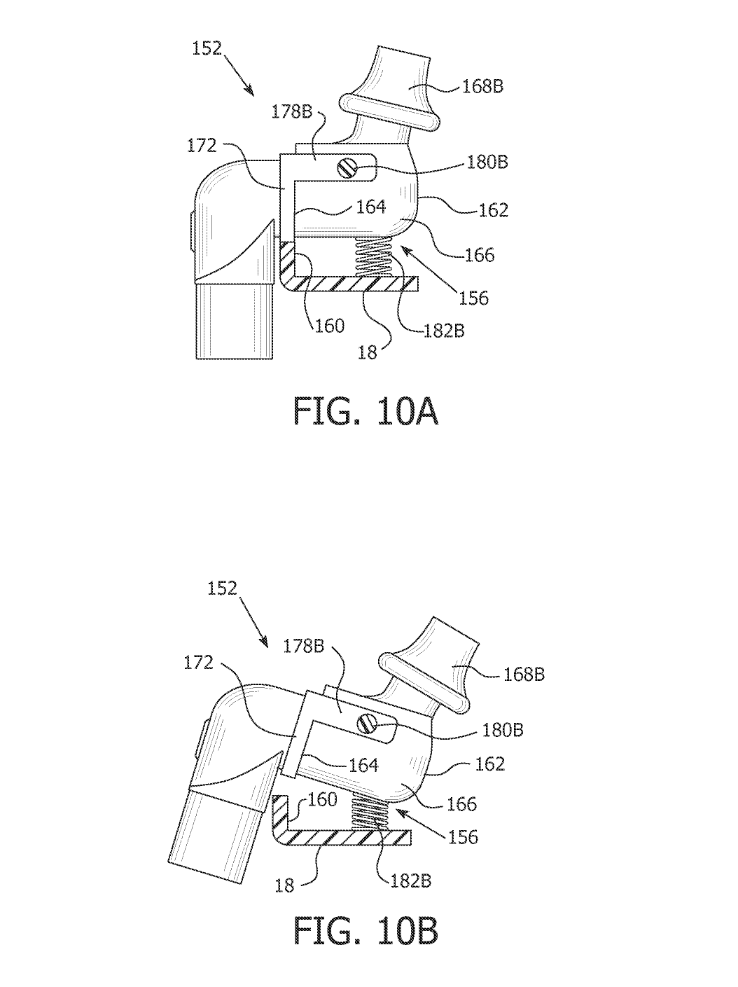

A system 100′ adapted to provide a regimen of respiratory therapy to a patient according to a further alternative exemplary embodiment is generally shown in FIGS. 10-13. System 100′ is similar to system 100, and includes a number of the same components as system 100, and like components are labeled with like reference numerals. However, as seen in FIGS. 7-8C, cushion assembly 106′, unlike cushion assembly 106, does not include post members 120A, 120B, and cushion assembly 106′ is thus not pivotably mounted to arms 20A, 20B of frame member 14. Instead, in this embodiment, cushion assembly 106′ is structured to float in between mounting portions 22A, 22B on spring members 126.

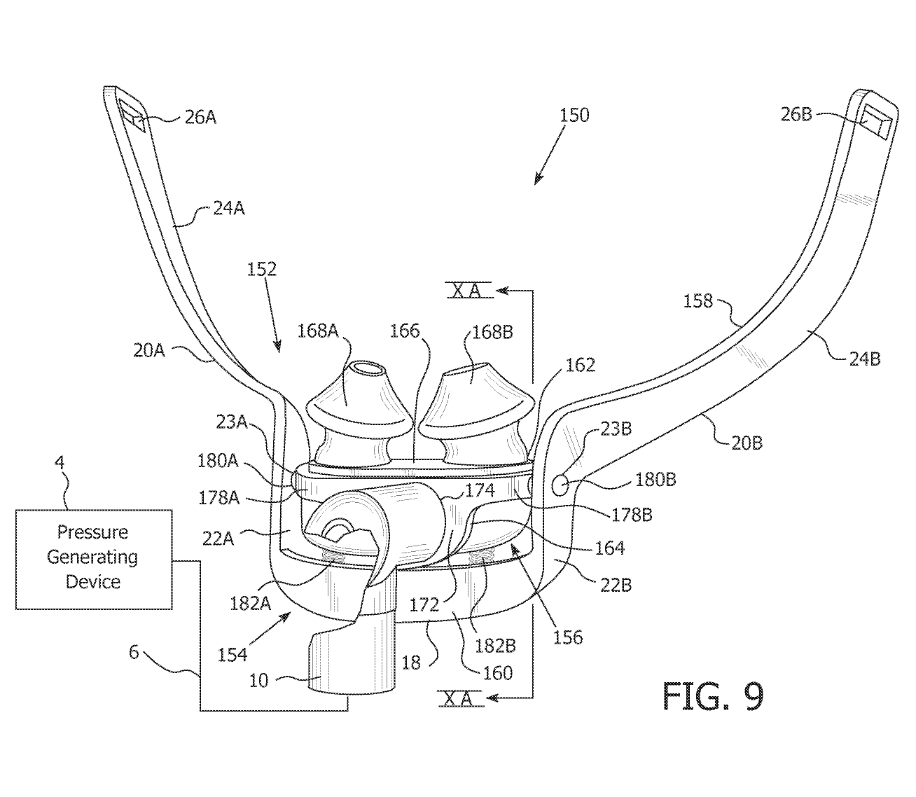

A system 150 adapted to provide a regimen of respiratory therapy to a patient according to still another alternative exemplary embodiment is generally shown in FIG. 9. System 150 includes a number of the same components as system 2, and like components are labeled with like reference numerals.

System 150 includes another alternative patient interface device 152 that is similar to patient interface device 8 (i.e., it includes fluid coupling conduit 10 and an automatically tilting cushion assembly/frame member combination). Patient interface device 152, however, includes another alternative patient sealing assembly 154 having an alternative cushion assembly 156 rotatably coupled to an alternative frame member 158 in the manner described in greater detail below. FIGS. 10A and 10B are cross-sectional views of a portion of alternative patient interface device 152 showing the operation of patient interface device 152 (described below).

As seen in FIG. 9, frame member 158 includes a number of the same parts as frame member 14. The central portion 18 of frame member 158, however, includes an upwardly extending stop portion 160, the function of which is described below.

Cushion assembly 156 includes a nasal cushion 162 structurally and fluidly coupled to a sub-frame member 164. In the illustrated embodiment, nasal cushion 162 is a “pillows” style nasal cushion made of flexible, cushiony, elastomeric material as described elsewhere herein. Alternatively, nasal cushion 162 may be a “cradle” style nasal cushion, or some other suitable nasal cushion configuration structured to engage the nose of the patient. As seen in FIGS. 9-10B, the exemplary pillows style nasal cushion 162 includes a main body portion 166 having nasal prongs 168A and 168B extending from a top side thereof.

In addition, sub-frame member 164, like sub-frame member 30 of FIGS. 1 and 2, is made of a rigid or semi-rigid material, such as, without limitation, an injection molded thermoplastic or silicone. Sub-frame member 164 includes a central support portion 172 having a central opening 174. As seen in FIGS. 9-10B, fluid coupling conduit 10 is coupled to a front side of central support portion 172 through opening 174. As also seen in FIGS. 9-10B, main body portion 166 of nasal cushion 162 is sealingly coupled to a rear side of central support portion 172 such that fluid coupling conduit 10 is in fluid communication with the interior of nasal cushion 162 through opening 174. This configuration allows the flow of breathing gas from pressure generating device 4 to be communicated to nasal cushion 162, and then to the airway of a patient (through the patient's nares which are engaged by nasal prongs 168A, 168B).

Sub-frame member 164 also includes pivot mounting portions 178A, 178B extending from opposite sides thereof. Each pivot mounting portion 178A, 178B includes a post member 180A, 180B.

Moreover, in this embodiment, a number of coiled (metal, e.g., steel) spring members 182 (182A and 182B in the illustrated embodiment) are provided in between the bottom surface of nasal cushion 162 at the rear end thereof and the top surface of central portion 18 of frame member 158. Also, as seen in FIG. 9, cushion assembly 156 is rotatably mounted to frame member 158 by inserting post members 180A, 180B through respective pivot openings 23A, 23B. When post members 180A, 180B are received in pivot openings 23A, 23B in this manner, cushion assembly 156 is able to rotate relative to frame member 158 about an axis through mounting portions 22A, 22B in a plane that is generally parallel to the extension portions 24A, 24B (i.e., toward and away from the patient's face as shown by the arrows in FIG. 14).

In addition, spring member(s) 182 provide rotational resistance to cushion assembly 156 relative to frame member 158. More particularly, cushion assembly 156 will have a preset original base position wherein spring member(s) 182 are in an un-compressed, relaxed state (with no force being applied to cushion assembly) as seen in FIG. 10A. In this state, the front end of the bottom surface of nasal cushion 162 will engage the top edge of stop portion 160. When a force is applied to nasal prongs 168A, 168B by the nose of the patient as a result of patient interface device 152 being donned by the patient, that force will work against the rotational resistance provided by spring member(s) 182, causing them to compress and bend as shown in FIG. 10B, and will cause cushion assembly 156 to automatically rotate (tilt) relative to frame member 158 (about the axis described above) to a tilted/loaded position that will accommodate the particular nose geometry of the patient. As seen in FIG. 10B, in this state, the front end of the bottom surface of nasal cushion 162 will lift off and be spaced from the top edge of stop portion 160. When that force is removed, the spring member(s) 182 will decompress and unbend and cause the cushion assembly 156 to automatically return to the original base position (unloaded). Again, in this state, the front end of the bottom surface of nasal cushion 162 will engage the top edge of stop portion 160 and prevent over rotation of cushion assembly 156.

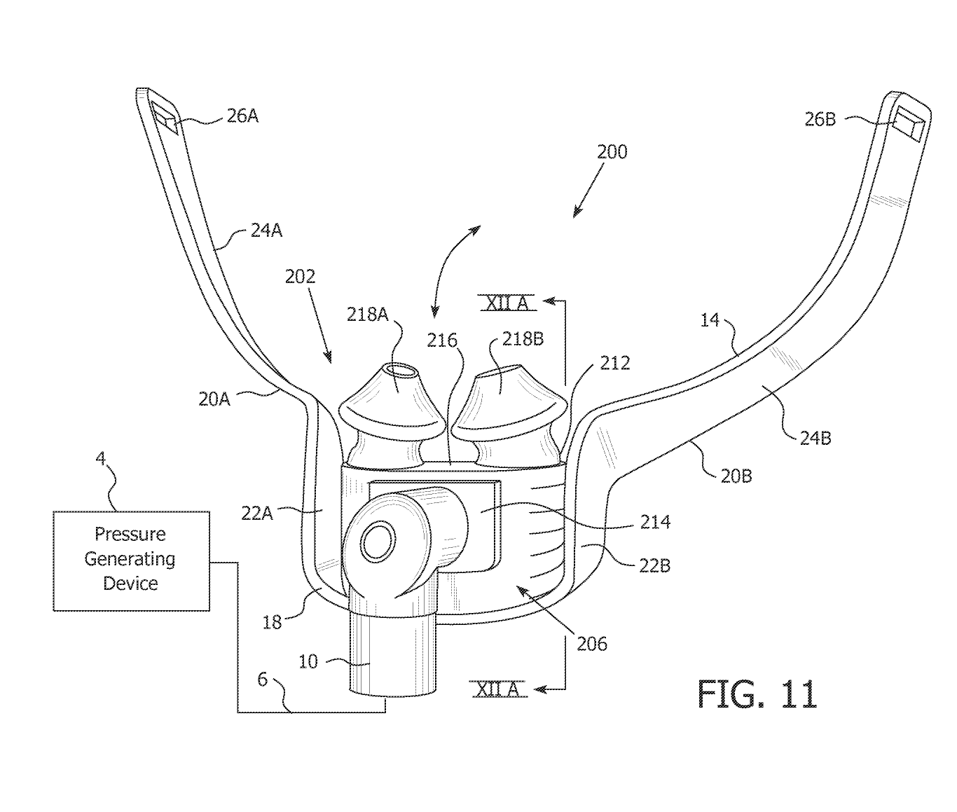

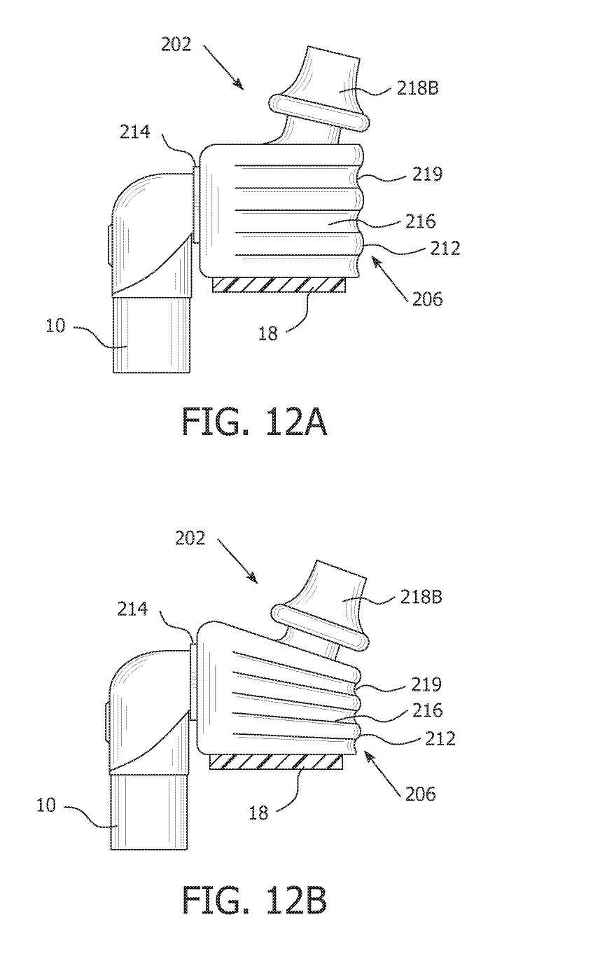

A system 200 adapted to provide a regimen of respiratory therapy to a patient according to an additional alternative exemplary embodiment is generally shown in FIGS. 11, 12A and 12B. System 200 is similar to system 100′ (like components are labeled with like reference numerals) in that the patient interface device 202 thereof has a cushion assembly 206 that does not include post members (like post members 120A, 120B), and cushion assembly 206 is thus not pivotably mounted to arms 20A, 20B of frame member 14. Instead, in this embodiment, cushion assembly 206 is supported on central portion 18 of frame member 14 and is structured to float in between mounting portions 22A, 22B. More specifically, cushion assembly 206 includes nasal cushion 212 having a main body portion 216 having nasal prongs 218A and 218B extending from a top side thereof. Cushion assembly 206 also includes a sub-frame 214. In addition, nasal cushion 212 includes a bellows section 219 on a rear side thereof extending from a top surface of main body portion 216 to a bottom surface of main body portion 216.

In operation, when patient interface device 202 is in use, the inside of nasal cushion 212 is under pressure and that pressure will tend to cause nasal cushion 212 to have an original base expanded state as shown in FIG. 12A. When a force is applied to nasal prongs 218A, 218B by the nose of the patient as a result of patient interface device 202 being donned by the patient, that force will work against the internal pressure just described, causing bellows section 219 to compress as shown in FIG. 12B. As a result, the tilt angle of nasal prongs 218A, 218B will automatically adjust to accommodate the particular nose geometry of the patient. When that force is removed, bellows section 219 will decompress and cause cushion assembly 206 to automatically return to the original expanded base position. In the exemplary embodiment, bellows section 219 has a cross-sectional thickness that is less than the cross-sectional thickness of the remainder of nasal cushion 212 to enable bellows section 219 to more easily collapse under the force provided by the nose of the patient as described above.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a cushion assembly including a nasal cushion, a first post member extending from a first side of the cushion assembly and a second post member extending from a second side of the cushion assembly;
a frame member having a central portion positioned below the nasal cushion, a first arm extending from a first side of the central portion and a second arm extending from a second side of the central portion, wherein the first arm includes a first mounting portion extending in a direction that is substantially normal to a top surface of the central portion and a second mounting portion extending in the direction that is substantially normal to the top surface of the central portion, wherein the first post member is rotatably coupled to the first mounting portion and the second post member is rotatably coupled to the second mounting portion such that the cushion assembly is rotatable with respect to the frame member about an axis extending through the first post member and the second post member; and
a rotational resistance mechanism coupled to the cushion assembly and the frame member, the rotational resistance mechanism providing resistance to rotation of the cushion assembly in a first direction toward a face of a patient when the patient interface device is donned by the patient and tending to bias rotation of the cushion assembly in a second direction opposite the first direction responsive to the cushion assembly being rotated in the first direction.

2. The patient interface device according to claim 1, wherein the cushion assembly further comprises a sub-frame member coupled to the nasal cushion, the first and second post members extending from opposite side of the sub-frame member.

3. The patient interface device according to claim 2, wherein the nasal cushion is fluidly coupled to a first side of the sub-frame member, the patient interface device including a fluid coupling conduit fluidly coupled to a second side of the sub-frame member such that the fluid coupling conduit is in fluid communication with the nasal cushion.

4. The patient interface device according to claim 2, wherein the rotational resistance mechanism comprises a first spring member having a first end coupled to the sub-frame and a second end coupled to the first arm, and a second spring member having a first end coupled to the sub-frame and a second end coupled to the second arm.

5. The patient interface device according to claim 4, wherein the first spring member and the second spring member each comprise a metal coiled spring.

6. The patient interface device according to claim 1, wherein the rotational resistance mechanism comprises an elastomeric tensioner member extending from a bottom surface of the nasal cushion, a distal end of the elastomeric tensioner member being coupled to the central portion of the frame member.

7. The patient interface device according to claim 6, wherein the central portion includes a slot, wherein a portion of the elastomeric tensioner member is received and held within the slot.

8. The patient interface device according to claim 7, wherein the portion of the elastomeric tensioner member received and held within the slot has a T-shape.

9. The patient interface device according to claim 1, wherein the cushion assembly further comprises a sub-frame member coupled to the nasal cushion, and wherein the sub-frame member includes a stop portion structured to engage the central portion to limit rotation of the cushion assembly.

10. The patient interface device according to claim 1, wherein the first and second post members extend directly from opposite sides of the nasal cushion.

11. The patient interface device according to claim 1, wherein the first mounting portion has a first pivot opening and the second mounting portion has a second pivot opening, wherein the first post member is received in the first pivot opening and the second post member is received in the second pivot opening.

12. The patient interface device according to claim 1, wherein the rotational resistance mechanism comprises one or more spring members coupled to and extending from a bottom surface of the nasal cushion, a distal end of each of the one or more spring members being coupled to the central portion of the frame member.

13. The patient interface device according to claim 12, wherein the one or more spring members extend from a rear end of the nasal cushion, wherein the central portion of the frame member has an upwardly extending stop portion structured to engage the bottom surface of the nasal cushion at a rear end thereof to limit rotation of the cushion assembly in the second direction, and wherein the bottom surface of the nasal cushion is structured to lift off the stop portion during rotation in the first direction.

14. The patient interface device according to claim 12, wherein the one or more spring members comprise a first one or more spring members coupled to and extending from a rear end of the nasal cushion and a second one or more spring members coupled to and extending from a front end of the nasal cushion opposite the rear end.

15. A patient interface device comprising:
a nasal cushion;
a frame member having a central portion positioned below the nasal cushion, a first arm extending from a first side of the central portion and a second arm extending from a second side of the central portion, wherein the first arm includes a first mounting portion extending in a direction that is substantially normal to a top surface of the central portion and a second mounting portion extending in the direction that is substantially normal to the top surface of the central portion; and
one or more spring members coupled to and extending from a surface of the nasal cushion, a distal end of each of the one or more spring members being coupled to the central portion of the frame member, the one or more spring members providing resistance to rotation of the nasal cushion in a first direction toward a face of a patient when the patient interface device is donned by the patient and tending to bias rotation of the cushion assembly in a second direction opposite the first direction responsive to the cushion assembly being rotated in the first direction.

16. The patient interface device according to claim 15, wherein the one or more spring members are coupled to and extend from a bottom surface of the nasal cushion.

17. The patient interface device according to claim 16, wherein the one or more spring members comprise a first one or more spring members coupled to and extending from a rear end of the nasal cushion and a second one or more spring members coupled to and extending from a front end of the nasal cushion opposite the rear end.

18. A method of adjusting a fit of a patient interface device having a cushion assembly including a nasal cushion, a first post member extending from a first side of the cushion assembly and a second post member extending from a second side of the cushion assembly, and a frame member having a central portion positioned below the nasal cushion, a first arm extending from a first side of the central portion and a second arm extending from a second side of the central portion, wherein the first arm includes a first mounting portion extending in a direction that is substantially normal to a top surface of the central portion and a second mounting portion extending in the direction that is substantially normal to the top surface of the central portion, wherein the first post member is rotatably coupled to the first mounting portion and the second post member is rotatably coupled to the second mounting portion, comprising:
providing resistance to rotation of the nasal cushion assembly in a first direction about an axis extending through the first arm and the second arm toward a face of a patient using a rotational resistance mechanism provided between the nasal cushion and the frame member; and
responsive to a force being applied to the nasal cushion by a nose of the patient, allowing the nasal cushion to rotate relative to the frame member in the first direction against the resistance to a tilted position, wherein in the tilted position the -rotational resistance mechanism biases the cushion assembly in favor of rotation in a second direction opposite the first direction.

19. (canceled)

20. The method according to claim 18, wherein the rotational resistance mechanism comprises a first spring member having a first end coupled to the nasal cushion and a second end coupled to the first arm, and a second spring member having a first end coupled to the nasal cushion and a second end coupled to the second arm.

21. The method according to claim 18, wherein the rotational resistance mechanism comprises an elastomeric tensioner member extending from a bottom surface of the nasal cushion, a distal end of the elastomeric tensioner member being coupled to the central portion of the frame member.

22. The method according to claim 18, wherein the rotational resistance mechanism comprises one or more spring members coupled to and extending from a bottom surface of the nasal cushion, a distal end of each of the one or more spring members being coupled to the central portion of the frame member.

23. The method according to claim 22, wherein the one or more spring members extend from a rear end of the nasal cushion, wherein the central portion of the frame member has an upwardly extending stop portion structured to engage the bottom surface of the nasal cushion at a rear end thereof to limit rotation of the nasal cushion in the second direction, and wherein the bottom surface of the nasal cushion is structured to lift off the stop portion during rotation in the first direction.

24. The method according to claim 22, wherein the one or more spring members comprise a first one or more spring members coupled to and extending from a rear end of the nasal cushion and a second one or more spring members coupled to and extending from a front end of the nasal cushion opposite the rear end.
